(19) United States
(12) Patent Application Publication
(10) Pub. No.: US 2024/0274269 A1
(43) Pub. Date: Aug. 15, 2024

(54) BEHAVIOR MODIFICATION SUPPORT DEVICE, METHOD AND PROGRAM

(71) Applicant: NIPPON TELEGRAPH AND TELEPHONE CORPORATION, Tokyo (JP)

(72) Inventors: Wataru AKAHORI, Musashino-shi, Tokyo (JP); Momoko NAKATANI, Musashino-shi, Tokyo (JP)

(73) Assignee: NIPPON TELEGRAPH AND TELEPHONE CORPORATION, Tokyo (JP)

(21) Appl. No.: 18/568,325

(22) PCT Filed: Jun. 10, 2021
(86) PCT No.: PCT/JP2021/022176

§ 371 Date: Dec. 8, 2023

(51) Int. Cl. G16H 20/70; A61B 5/16

(52) U.S. Cl. CPC G16H 20/70; A61B 5/165

(57) ABSTRACT

For each group, relationship information indicating a strength of a relationship between a plurality of users belonging to the group is acquired. At the same time, for each group, psychological state evaluation information in which psychological states of the plurality of users belonging to the group are reflected is acquired. In this state, for each group, a first user of which the acquired psychological state evaluation information does not satisfy a preset first condition is selected as a user who is a support target of the behavior change, and, on the basis of the relationship information, a second user of which a strength of a relationship with the first user satisfies a preset second condition is selected as a user who is a request destination of support of the behavior change for the first user. First message information including information for requesting support of the behavior change is generated.

### TECHNICAL FIELD

One aspect of the present invention relates to a behavior change support device, a method, and a program used to support a behavior change of a group, for example.

### BACKGROUND ART

Conventionally, for example, many technologies for promoting a behavior change of an individual by using life log data have been proposed. On the other hand, regarding a group such as a sports team or a company, since the group has various human relationships inside unlike an individual, it is not clear whether there is an effect even if the technology for promoting a behavior change of an individual is applied as it is.

Therefore, as a technology for promoting a behavior change of a group, a technology for supporting a behavior change of a group by combining elements of “competition” and “collaboration” has been proposed (for example, refer to Non Patent Literature 1).

### CITATION LIST

### Non Patent Literature

Non Patent Literature 1: Yuuki Nishiyama, Tadashi Okoshi, Takuro Yonezawa, Jin Nakazawa, Kazunori Takashio, and Eideyuki Tokuda, “Encouraging Team Behavior Modification Using Lifelog”, Journal of Information Processing Society of Japan, 56(1), 349-361. (January 2015)

### SUMMARY OF INVENTION

### Technical Problem

However, for example, even if the elements of “competition” and “collaboration” are simply combined as in the technology disclosed in Non Patent Literature 1, it is difficult to obtain a sufficient effect since individuals belonging to a group have different psychological states and relationships.

The present invention has been made in view of the above circumstances, and in one aspect, an object of the present invention is to provide a technique capable of effectively supporting a behavior change of a group in consideration of a state of each individual belonging to the group.

### Solution to Problem

In order to solve the above problems, an aspect of a behavior change support device or a method according to the present invention is a behavior change support device that supports a behavior change for at least one of a plurality of groups to which a plurality of users belongs, in which first, for each of the groups, relationship information indicating a strength of a relationship between the plurality of users belonging to the group is acquired and stored. At the same time, for each of the groups, psychological state evaluation information in which psychological states of the plurality of users belonging to the group are reflected is acquired. In this state, for each of the groups, a first user of which the acquired psychological state evaluation information does not satisfy a preset first condition is selected as a user who is a support target of the behavior change, and, on the basis of the relationship information, a second user of which a strength of a relationship with the first user satisfies a preset second condition is selected as a user who is a request destination of support of the behavior change for the first user. First message information including information for requesting support of the behavior change is generated, and the generated first message information is transmitted to a user terminal used by the second user.

Here, the behavior change includes changing speech and a behavior and changing feelings in addition to actually physically changing a behavior.

According to one aspect of the present invention, for example, a user having a strong relationship with a user having a poor psychological state in a group and having a good psychological state is selected as a suitable user for talking to the user having the poor psychological state, and a request message for making a request for talking to the user having the poor psychological state is transmitted to a user terminal of the suitable user. Therefore, it is possible to perform effective behavior change support for a user having a poor psychological state from a user having a strong relationship with the user in the group.

### Advantageous Effects of Invention

That is, according to one aspect of the present invention, it is possible to provide a technique capable of effectively supporting a behavior change of a group in consideration of a state of each individual belonging to the group.

### BRIEF DESCRIPTION OF DRAWINGS

FIG. 1 is a diagram illustrating an example of a system including a server device functioning as a behavior change support device according to an embodiment of the present invention.

FIG. 2 is a block diagram illustrating a hardware configuration of the server device illustrated in FIG. 1.

FIG. 3 is a block diagram illustrating a software configuration of the server device illustrated in FIG. 1.

FIG. 4 is a diagram illustrating an example of data indicating a relationship between individuals (users) belonging to a group, stored in a relationship data storage unit of the server device illustrated in FIG. 3.

FIG. 5 is a diagram illustrating an example of data indicating a correspondence relationship between a user and a psychological state evaluation value thereof, stored in a data storage unit 3 of the server device illustrated in FIG. 3.

FIG. 6 is a diagram illustrating an example of registration results of a group, and a psychological state evaluation value, a dialogue state evaluation value, a degree of goal achievement, and text data related to a goal for each user belonging to the group, which are stored in a behavior change data storage unit of the server device illustrated in FIG. 3.

FIG. 7 is a flowchart illustrating a processing procedure and processing details of a behavior change support process executed in a control unit of the server device illustrated in FIG. 3.

### DESCRIPTION OF EMBODIMENTS

Hereinafter, embodiments according to the present invention will be described with reference to the drawings.

### One Embodiment

### Configuration Example

### (1) System

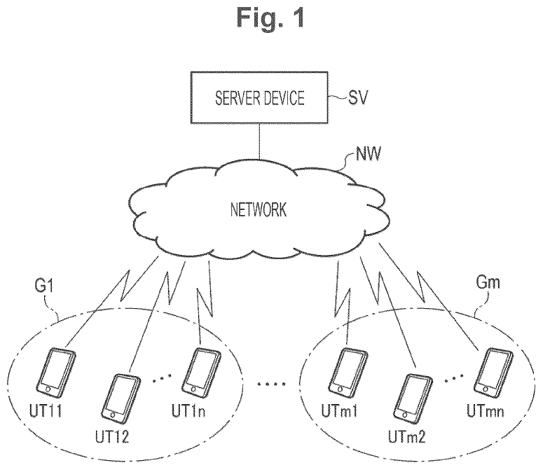

FIG. 1 is a diagram illustrating an example of a system including a server device functioning as a behavior change support device according to an embodiment of the present invention.

In FIG. 1, G1 to Gm denote groups that are targets of a behavior change (hereinafter, also referred to as groups), and a plurality of individuals (hereinafter, also referred to as users) belong to each of the groups G1 to Gm. The respective users own information terminals UT11 to UT1n, . . . , and UTm1 to UTmn (hereinafter collectively referred to as user terminals UT).

The user terminal UT includes, for example, a portable information communication terminal such as a smartphone, a tablet terminal, or a laptop personal computer, and can transmit information data between the user terminals UT and a server device SV via a network NW in addition to a creation process, an editing process, and the like of various types of data such as document data and chart data. In order to transmit the information data, the user terminal UT includes a mailer, a browser, and an SNS application. As the user terminal UT, a desktop personal computer or the like may be used in addition to the portable information communication terminals described above.

The network NW includes, for example, a wide area network having the Internet as a core and an access network for accessing the wide area network. As the access network, for example, a public communication network using a wired or wireless network, a local area network (LAN) using a wired or wireless network, or a cable television (CATV) network is used.

### (2) Server Device SV

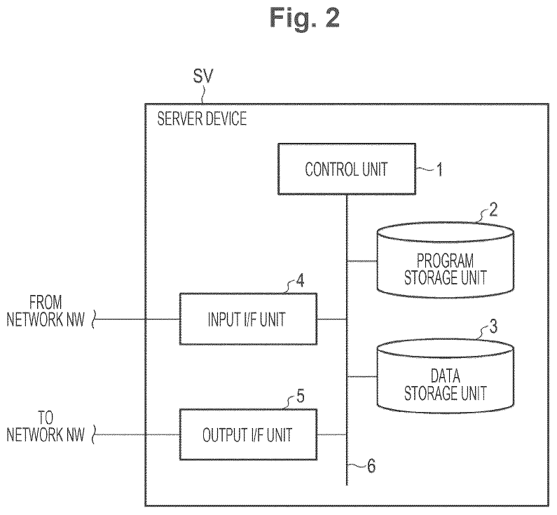

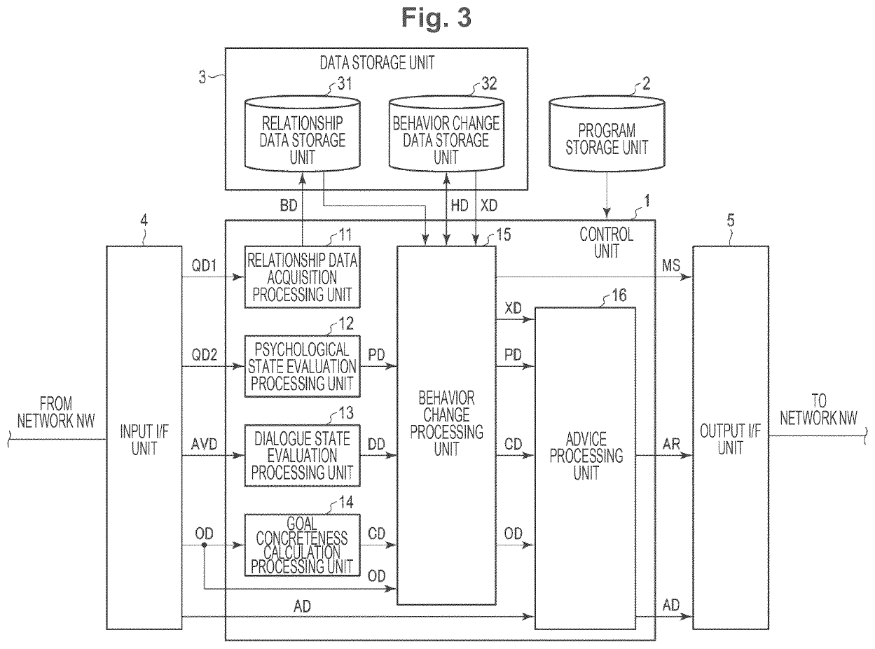

FIGS. 2 and 3 are block diagrams respectively illustrating a hardware configuration and a software configuration of the server device SV.

The server device SV includes, for example, a server computer disposed in a web or a cloud, and includes a control unit 1 using a hardware processor such as a central processing unit (CPU). The server device SV is configured such that a storage unit including a program storage unit 2 and a data storage unit 3, an input I/F unit 4, and an output I/F unit 5 are connected to the control unit 1 via a bus 6.

Both the input I/F unit 4 and the output I/F unit 5 have a communication interface, and receive and transmit information data from the user terminal UT by using a communication protocol defined by the network NW under the control of the control unit 1.

For example, the program storage unit 2 is configured by combining a nonvolatile memory capable of writing and reading data as needed, such as a hard disk drive (HDD) or a solid state drive (SSD), and a nonvolatile memory such as a read only memory (ROM) as a storage medium, and stores an application program necessary for executing various control processes according to an embodiment of the present invention, in addition to middleware such as an operating system (OS).

The data storage unit 3 is configured, for example, by combining a nonvolatile memory capable of writing and reading data as needed, such as an HDD or an SSD, with a volatile memory, such as a random access memory (RAM), as storage media, and includes a relationship data storage unit 31 and a behavior change data storage unit 32 in addition to an attribute information storage unit that stores attribute information regarding a group and a user as a main storage unit necessary for carrying out an embodiment of the present invention.

In the attribute information storage unit, for example, information indicating an attribute of a group is stored in association with a group ID, and information indicating attributes of a plurality of users belonging to the group is stored in association with user IDs of the users. The attribute of the group includes, for example, information indicating a genre of the group such as work, sports, or hobby, and the attribute of the user includes, for example, contact information such as a name or an e-mail address of the user.

The relationship data storage unit 31 stores, for each group, information indicating the strength of social human relationships between a plurality of users belonging Lo the group. The strength of the human relationship is characterized by, for example, an interaction frequency or an interaction accumulation time, the strength of emotion, closeness (mutual reliability), and the frequency and details of help, and is quantified on the basis of, for example, answer information of a user to a questionnaire.

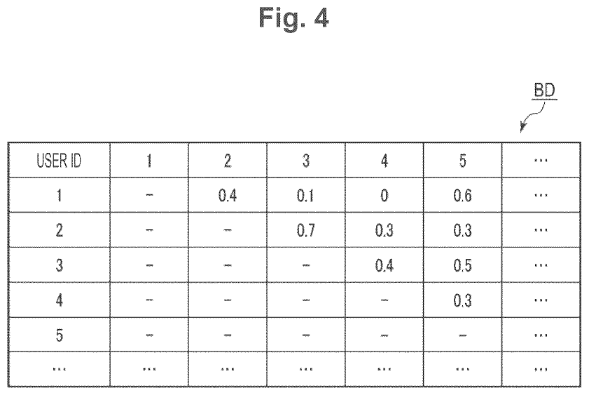

FIG. 4 illustrates an example of information indicating the strength of a relationship between users belonging to a certain group, stored in the relationship data storage unit 31.

The behavior change data storage unit 32 is used to store a psychological state evaluation value of a user, a goal concreteness, and text data for the goal in association with a group ID and a user ID, and to store a dialogue state evaluation value for each group.

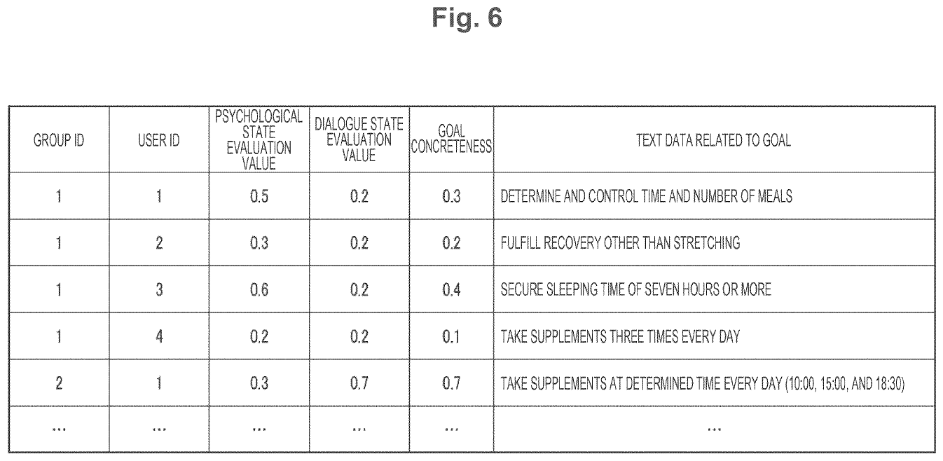

FIG. 6 illustrates an example of a psychological state evaluation value, the goal concreteness, a goal concreteness, and text data related to the goal of each user belonging to a certain group, and a dialogue state evaluation value of the group, stored in the behavior change data storage unit 32.

The control unit 1 includes a relationship data acquisition processing unit 11, a psychological state evaluation processing unit 12, a dialogue state evaluation processing unit 13, a goal concreteness calculation processing unit 14, a behavior change processing unit 15, and an advice processing unit 16 as processing functions according to an embodiment of the present invention. Each of these processing units 11 to 16 is realized by causing a hardware processor of the control unit 1 to execute a program stored in the program storage unit 2.

For example, the relationship data acquisition processing unit 11 performs a process of transmitting questionnaire data to each user terminal UT, obtaining a strength BD of a relationship between the users on the basis of answer data QD1, and storing the strength BD in the relationship data storage unit 31. As means for obtaining the strength BD of the relationship between the users, for example, information indicating an interaction frequency or an interaction accumulation time between the users may be acquired from a use history of a communication tool of each user, and the strength BD of relationship between the users may be estimated on the basis of the acquired information.

For example, the psychological state evaluation processing unit 12 performs a process of obtaining an evaluation value PD indicating a psychological state of each user by transmitting questionnaire data to each user terminal UT and acquiring answer data QD2. As means for obtaining the evaluation value PD of the psychological state of the user, for example, a means for acquiring vital data indicating heart rate variability (HRV) of the user from the user terminal UT, calculating an average value of HRV in a past predetermined period on the basis of the acquired vital data, and obtaining psychological state evaluation value PD of the user on the basis of the average value may be used. The psychological state evaluation processing unit 12 temporarily stores the calculated psychological state evaluation value PD in the data storage unit 3, and then passes the psychological state evaluation value PD to the behavior change processing unit 15.

For example, the dialogue state evaluation processing unit 13 acquires dialogue audio data or dialogue video data AVD of each user in the group from the user terminal UT. By analyzing the acquired data AVD, a process of calculating a dialogue state evaluation value DD indicating the degree of activity (degree of excitement) of the dialogue in the group is performed. An example of a process of obtaining the dialogue state evaluation value DD will be described in an operation example.

For example, the goal concreteness calculation processing unit 14 performs a process of acquiring text data OD related to a goal from the user terminal UT for each user, and calculating the goal concreteness CD of the user by analyzing the acquired text data OD related to the goal. The goal concreteness calculation processing unit 14 calculates a goal group characteristic representing features of the goal of a group on the basis of the acquired text data OD related to the goal. An example of a goal group characteristic calculation process will be described in an operation example.

The behavior change processing unit 15 has, for example, the following processing functions.

(1) A process of respectively acquiring the psychological state evaluation value PD and the goal concreteness CD of each user from the psychological state evaluation processing unit 12 and the goal concreteness calculation processing unit 14, acquiring the dialogue state evaluation value DD of the group from the dialogue state evaluation processing unit 13, and storing the acquired data PD, CD, and DD in the behavior change data storage unit 32 in association with the group ID and the user ID

(2) A process of acquiring the text data OD related to the goal sent from each user terminal UT from the input I/F unit 4, and storing the acquired text data OD related to the goal in the behavior change data storage unit 32 in association with the user ID

(3) A process of reading the dialogue state evaluation value DD of the group from the behavior change data storage unit 32 and determining the presence or absence of a dialogue for each group on the basis of the dialogue state evaluation value DD

(4) A process: For a group for which it is determined in (3) that there is no dialogue, the psychological state evaluation values PD of a plurality of users belonging to the group are read from the behavior change data storage unit 32, and a user who is a behavior change promotion target in the group is selected on the basis of the psychological state evaluation values PD. Subsequently, the relationship data BD indicating the strength of the relationship between the users is read from the relationship data storage unit 31, and a user of which value indicating the strength of the relationship with the user who is a behavior change promotion target is equal to or greater than a threshold value is selected as a user to be a request destination of support of a behavior change for the user who is a behavior change promotion target on the basis of the relationship data BD. A behavior change support request message for the selected request destination user is generated, and the generated behavior change support request message is transmitted from the output I/F unit 5 to the corresponding user terminal UT.

(5) A process: For a group for which it is determined in (3) that there is no dialogue, the goal concreteness CD of each user belonging to the group is read from the behavior change data storage unit 32, and a user of which goal concreteness CD is less than a threshold value of the goal concreteness of the group is selected as a user who is a behavior change promotion target. A user of which goal concreteness CD in the same group is equal to or more than the threshold value is selected as a reference user, a behavior change promotion message MS including the goal concreteness CD of the selected reference user and text data OD related to the goal is generated, and the generated behavior change promotion message MS is transmitted from the output I/F unit 5 to the user terminal UT of the user who is a behavior change promotion target.

(6) A process: For a group for which it is determined in (3) that there is a dialogue, a group in which the dialogue state evaluation value DD is smaller than a dialogue state evaluation threshold value common to all groups, that is, the dialogue activity is low is extracted as a group that is a behavior change promotion target. The goal group characteristic included in the goal concreteness CD of each group is read from the behavior change data storage unit 32, and another group in which the goal group characteristic is similar to a goal group characteristic of the group that is a behavior change promotion target and the dialogue state evaluation value DD is equal to or more than the dialogue state evaluation threshold value is selected. Next, the behavior change promotion message MS including the goal concreteness CD of the selected other group and the text data OD related to the goal, and the generated behavior change promotion message MS is transmitted from the output I/F unit 5 to the user terminal UT of each user belonging to the group that is a behavior change promotion target.

The advice processing unit 16 has the following processing functions.

(1) A process: For each user, the psychological state evaluation value PD, the text data OD related to the goal, and the goal concreteness CD are acquired from the behavior change data storage unit 32 or the behavior change processing unit 15, and an advice request message AR for advice for promoting a behavior change for each user is generated on the basis of the acquired data PD, OD, and CD. The generated advice request message AR is transmitted from the cutout I/F unit 5 to the user terminals UT of other users in the group.

(2) A process: When an advice message AD is returned from the user terminal UT of another user with respect to the advice request message AR, the advice message AD is received via the input I/F unit 4. The received advice message AD is transmitted from the output I/F unit 5 to be transferred to the user terminal UT of the user who has requested the advice.

### Operation Example

Next, an example of a behavior change support operation performed by the server device SV configured as described above will be described.

A description will be made on the assumption that attribute information of each group and a plurality of users belonging to a group is stored in advance in the attribute information storage unit in the data storage unit 3. The attribute information includes, for example, a group ID and attribute information of an affiliated work organization (for example, information indicating a genre such as work, sports, or hobby), and a user ID and attribute information thereof (for example, contact information such as a name and an e-mail address), but is not limited thereto.

### (1) Acquisition of Relationship Data

Under the control of the relationship data acquisition processing unit 11, the control unit 1 of the server device SV acquires, for each group, data indicating a relationship between a plurality of users belonging to the group as follows.

That is, the relationship data acquisition processing unit 11 first transmits questionnaire data to the user terminal UT of a target user. The questionnaire data includes, for example, a question for asking, for each user, each other user in the same group about, for example, an interaction frequency, an interaction accumulation time, the strength of emotion, the degree of intimacy, and the degree of help. In this case, a plurality of choices corresponding to the strength of the relationship are presented for each question item in an answer field to the question, and a corresponding choice may be selected from these choices.

Instead of using the questionnaire, for example, the relationship data acquisition processing unit 11 may acquire data indicating a use history of the communication tool from each user terminal UT, calculate information indicating an interaction frequency or an interaction time between the users from the acquired use history data, and estimate the strength of the relationship between the users on the basis of the result.

The relationship data acquisition processing unit 11 generates the acquired strength of the relationship between the users as a numerical value between, for example, 0 and 1, and stores the numerical value in the relationship data storage unit 31 of the data storage unit 3. The process of acquiring data indicating the strength of the relationship between the users is performed, for example, periodically or every time a group organization is changed.

### (2) Acquisition of Psychological State Evaluation Value

Under the control of the psychological state evaluation processing unit 12, the control unit 1 of the server device SV executes, for each group, a process of acquiring an evaluation value of a psychological state of each user belonging to the group as follows.

That is, the psychological state evaluation processing unit 12 transmits questionnaire data to the user terminal UT of a target user, for example, similarly to the relationship data acquisition process described above. The questionnaire data includes, for example, a question for asking about the comfort in the group, an execution result of a task, and the like. Also in this case, a plurality of choices corresponding to a level of the psychological state are presented for each question item in an answer field to the question, and a corresponding choice may be selected from these choices.

The psychological state evaluation processing unit 12 receives answer data of the user to the questionnaire from the user terminal UT, evaluates a psychological state of the user on the basis of the received answer data, and temporarily stores the evaluation value in the data storage unit 3.

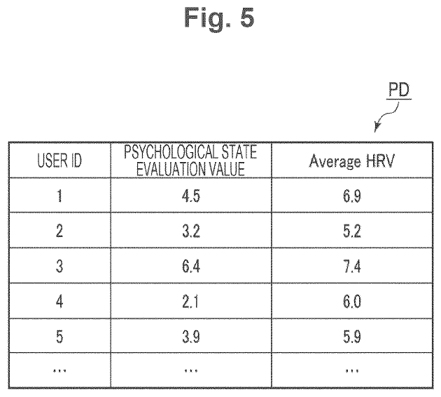

The psychological state evaluation processing unit 12 may acquire vital data indicating heart rate variability, which is an index of the autonomous adjustment activity of the user, from the user terminal UT, for example, calculate an average value of HRV in a past predetermined period on the basis of the acquired vital data, and store the average value as a psychological state evaluation value of the user on the basis of the value. FIG. 5 illustrates an example of a storage result of the psychological state evaluation value in this case.

The process of acquiring the psychological state evaluation value is executed periodically or every time an event in the group is performed, for example.

### (3) Acquisition of Dialogue State Evaluation Value of Croup

Under the control of the dialogue state evaluation processing unit 13, the control unit 1 of the server device SV executes, for each group, a process of acquiring a dialogue state evaluation value indicating the degree of activity of the dialogue of the group as follows.

That is, the dialogue state evaluation processing unit 13 acquires dialogue audio data or dialogue video data of the plurality of users in the group from each user terminal UT for each group. By analyzing the acquired data associated with the dialogue, a dialogue state evaluation value indicating the degree of activity (degree of excitement) of the dialogue in the group is calculated.

For example, the dialogue state evaluation processing unit 13 extracts feature amounts such as energy and pitch of voice, and an utterance rate from the acquired dialogue audio data of the user, and extracts feature amounts such as head or hand movements and a person's pose from the dialogue video data. The degree of excitement of the dialogue is obtained on the basis of each of the extracted feature amounts by using, for example, a machine learning model. The dialogue state evaluation processing unit 13 may extract topics from the dialogue content by using morphological analysis or the like, and obtain a dialogue state evaluation value on the basis of the extracted topic types and the number thereof.

The dialogue state evaluation value is generated as a numerical value, for example, in a range of “0” in a case where there is no dialogue for a certain period of time, or in a range of, for example, 0 to 1 in a case where there is a dialogue.

### (4) Calculation of Goal Concreteness

The control unit 1 of the server device SV calculates, for each group, the concreteness of the goal of each user belonging to the group under the control of the goal concreteness calculation processing unit 14, and executes a process of obtaining a goal group characteristic representing features of a goal of the group as follows.

That is, the goal concreteness calculation processing unit 14 first acquires text data related to the goal for each user from the user terminal UT, and calculates the concreteness of the goal of the user by analyzing the acquired text data related to the goal. For example, the text data related to the goal is morphologically analyzed, the degree of importance, a goal numerical value, and the like of a word or a phrase representing the behavior content are extracted, and the goal concreteness is calculated on the basis of the result. The goal concreteness is generated as a numerical value in a range of 0 to 1, for example.

The goal concreteness calculation processing unit 14 calculates a goal group characteristic representing a characteristic of a goal of a group on the basis of the acquired text data related to the goal.

The goal group characteristic is obtained, for example, by calculating the importance of each word included in the text from the text data related to the goal by using a term frequency-inverse document frequency (TD-IDF) method and creating a vector having all the words included in the entire text as dimensions for each goal.

The goal concreteness calculation processing unit 14 temporarily stores data indicating the calculated goal concreteness of each user and similarity of the goal group characteristic in the data storage unit 3, for example, and then passes the data to the behavior change processing unit 15.

### (5) Transmission of Behavior Change Promotion Message

The control unit 1 of the server device SV executes a support process for promoting a behavior change of a group under the control of the behavior change processing unit 15 as follows.

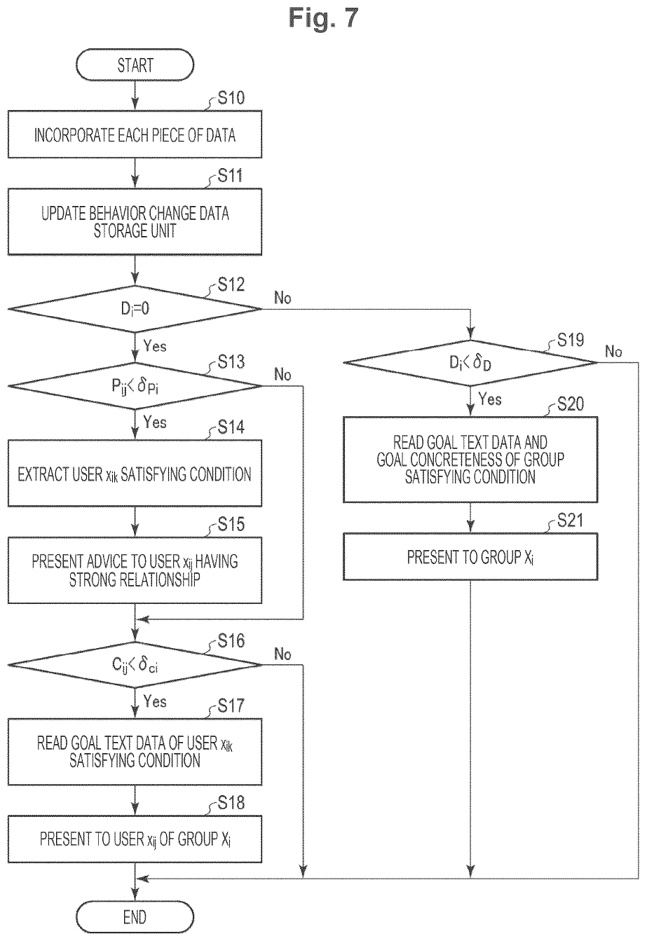

FIG. 7 is a flowchart illustrating a procedure and processing details of a support process executed by the behavior change processing unit 15.

### (5-1) Incorporation of Data Such as Various Evaluation Values

First, in step S10, the behavior change processing unit 15 acquires, for each group, the psychological state evaluation value PD and the goal concreteness CD of each user belonging to the group from the psychological state evaluation processing unit 12 and the goal concreteness calculation processing unit 14, respectively. The behavior change processing unit 15 acquires the dialogue state evaluation value DD of each group from the dialogue state evaluation processing unit 13. In step S11, the behavior change processing unit 15 stores the acquired psychological state evaluation value PD and goal concreteness CD of each user, and the acquired dialogue state evaluation value DD of each group in the behavior change data storage unit 32 in association with a group ID and a user ID.

The behavior change processing unit 15 also incorporates the text data OD related to the goal transmitted from each user terminal UT. The incorporated text data OD related to the goal is stored in the behavior change data storage unit 32 in association with the user ID. FIG. 6 illustrates an example of each piece of data stored in the behavior change data storage unit 32 as described above.

Hereinafter, a group is denoted by Xi (where i=1, 2, . . . , and I), a user belonging to the group Xi is denoted by xij (where j=1, 2, . . . , and Ji), a psychological state evaluation value of the user xij is denoted by Pij (where 0≤Pij≤1), a dialogue state evaluation value of the group Xi is denoted by Di (where 0≤Di≤1), a goal concreteness of the user xij is denoted by Cij (where 0≤Cij≤1), and an advice from the user xij to the user xik is denoted by Aijk (where j and k are different). A threshold value for the psychological state evaluation value of the group Xi is denoted by δpi, a threshold value for the dialogue state evaluation values of all the groups is denoted by δD, and a threshold value for the goal concreteness of the group Xi is denoted by δCi.

### (5-2) Determination of Presence or Absence of Dialogue in Group

When the incorporation of each data is ended, in step S12, first, the behavior change processing unit 15 reads the dialogue state evaluation value Di of the group Xi from the behavior change data storage unit 32 and determines whether or not the dialogue state evaluation value Di is 0, that is, whether or not there is a dialogue in the group Xi.

### (5-3) Support of Behavior Change of User in Bad Condition

When it is determined in step S12 that there is no dialogue in the group Xi, in step S13, first, the behavior change processing unit 15 compares the psychological state evaluation value Pij of each user belonging to the group Xi with the threshold value δpi for the psychological state evaluation value of the group Xi. The threshold value δpi in this case is set to, for example, an average value of the psychological state evaluation values Pij of all the users xij in the group Xi. As a result of this comparison, the user xik of which psychological state evaluation value Pij is smaller than the threshold value δpi is extracted as a user who is not in a good condition, that is, a user who is a behavior change promotion target in step S14.

Next, in step S15, the behavior change processing unit 15 reads the relationship data BD indicating the strength of the relationship between the users from the relationship data storage unit 31, and selects another user xij of which a value indicating the relationship with the user xik who is not in a good condition is equal to or more than the threshold value, that is, having a strong relationship, on the basis of the relationship data BD. Among the users xij, a user having the highest similarity between the user xik who is in a band condition and the text data related to the goal may be selected. The behavior change processing unit 15 further selects a user of which psychological state evaluation value Pij is the threshold value δpi from among the selected users xij.

As a result, a user having a strong relationship with the user who is not in a good condition in the group and having a good psychological state is selected as a suitable user in taking to the user who is not in a good condition.

Next, the behavior change processing unit 15 generates a request message MS for making a request for talking to the user xik who is not in a good condition. The generated request message MS is transmitted from the output I/F unit 5 to the user terminal UT of the user suitable for talking.

As a result, the support of the behavior change is provided to the user who is not in a good condition from the user having a strong relationship with the user in the group, and thus the support of the behavior change can be effectively provided to the user who is not in a good condition.

(5-4) Support of Behavior Change of Group with No Dialogue

In step S16, the behavior change processing unit 15 further reads the goal concreteness Cij of each user xij belonging to the group Xi for which it is determined that there is no group dialogue from the behavior change data storage unit 32. The goal concreteness Cij is compared with the threshold value δci for the goal concreteness of the group Xi. The threshold value δci in this case is set to, for example, an average value of the goal concreteness Cij of all the users xij in the group Xi. As a result of the comparison, a user of which goal concreteness Cij is less than the threshold value δci is extracted as a user that is a behavior change promotion target.

Subsequently, the behavior change processing unit 15 selects another user of which goal concreteness Cik is equal to or more than the threshold value δci in the same group, and reads the text data OD and the goal concreteness Cik related to the goal of the selected user from the behavior change data storage unit 32. A behavior change promotion message MS including the read goal concreteness Cik and text data OD related to the goal is generated, and the generated behavior change promotion message MS is transmitted from the output I/F unit 5 to the user terminal UT of the user xij who is a behavior change promotion target in step S18.

As a result, the text data related to the goal of the user having the high goal concreteness in the same group and the goal concreteness are presented to the user having the low goal concreteness in the group, that is, the user of which goal setting is ambiguous, and thus, it is possible to prompt the user having the low goal concreteness to review the goal setting.

### (5-5) Support of Behavior Change of Group Having Dialogue

For the group Xi for which it is determined in step S12 that there is a dialogue, the behavior change processing unit 15 compares the dialogue state evaluation value Di with the dialogue state evaluation threshold value δD common to all the groups in step S19. In this case, the threshold value δD is set to, for example, an average value of the dialogue state evaluation values of all the groups. As a result of this comparison, a group in which the dialogue state evaluation value Di is smaller than the dialogue state evaluation threshold value δD, that is, a group Xk in which the dialogue activity (degree of excitement) is low is extracted as a group that is a behavior change promotion target.

Next, in step S20, the behavior change processing unit 15 reads the goal group characteristic included in the goal concreteness Cij of each group Xi from the behavior change data storage unit 32. A group in which the goal group characteristic is similar to the group Xk that is a behavior change promotion target and the dialogue state evaluation value Di is equal to or greater than the dialogue state evaluation threshold value δD is selected. That is, a group having similar characteristics of the goal as a group and having a high dialogue activity is selected as a reference group that promotes a behavior change of the group Xk. The group having similar goal group characteristics is extracted by, for example, computing cosine similarity of vectors between the groups and comparing the computed cosine similarity with a threshold value.

Subsequently, the behavior change processing unit 15 reads the text data OD related to the goal and the goal concreteness of the selected similar group from the behavior change data storage unit 32, and generates a behavior change promotion message MS including these pieces of information. In step S21, the generated behavior change promotion message MS is transmitted from the output I/F unit 5 to the user terminal UT of each user xkj belonging to the group Xk that is a behavior change promotion target.

In this case, for example, the behavior change promotion message MS such as “Your group's dialogue does not seem to be very active. On the other hand, other groups seem to be actively engaged in dialogue. In this group, for example, ‘taking supplementations at predetermined time every day (10:00, 15:00, and 18:30)’ is set to the concreteness of “0.7”, ‘in a case of a practice being late, eating more carbohydrates (three rice balls) in supplementations after practice to reduce an amount of carbohydrates for dinner’ is set to a concreteness of “0.8”, and ‘eating warm vegetables such as broccoli and asparagus daily’ is set to a concreteness of “0.6”. Let's discuss these goals again in the group and set them.” is used.

By performing such behavior change support, text data related to a goal of a group having similar goal group characteristics among other groups in which the dialogue activity is equal to or more than the average and a goal concreteness are presented to each user of the group in which the dialogue activity (degree of excitement) is less than the average although the dialogue is being performed. Consequently, in a group having a low dialogue activity, it is possible to promote a behavior change as a group with reference to information of another group having a high dialogue activity.

### (6) Presentation of Advice

Under the control of the advice processing unit 16, the control unit 1 of the server device SV executes advice processing for a group in which a behavior change is required and a user thereof, for example, as follows.

That is, first, the advice processing unit 16 acquires, for each group, the psychological state evaluation value PD of each user belonging to the group, the text data OD related to the goal, and the goal concreteness CD thereof from the behavior change data storage unit 32 via the behavior change processing unit 15. A user estimated to need an advice from another user is selected from among the users on the basis of the acquired data PD, OD, and CD. Subsequently, the advice processing unit 6 generates advice request message AR for asking another user to give advice to the user, and transmits the generated advice request message AR from the output I/F unit 5 to the user terminals UT of the other users in the group.

In a case where the advice message AD is returned from the user terminal UT of another user to the advice request message AR, the advice processing unit 16 receives the advice message AD via the input T/F unit 4. The received advice message AD is transferred from the output I/F unit 5 to the user terminal UT of the user who needs the advice.

For example, a case will be described as an example in which advice from other users is requested for “Hiroshi Tanaka” registered with “running 10 km every day to lose 10 kg weight” as text data related to the current goal, “0.3” as a goal concreteness, and “0.2” as a psychological state evaluation value.

In this case, the advice processing unit 16 generates a message such as “Ichiro Tanaka set a goal, ‘running 10 km every day to lose 10 kg weight’, with a goal concreteness of 0.3 and a psychological state of 0.2. Do you have any advice to Ichiro Tanaka?” as the advice request message AR and transmits the advice request message AR to other users.

It is assumed that another user returns, for example, “Mr./Ms. Tanaka, it may be a difficult time, but please do your best. If it is difficult to run 10 km at a time, it is also recommended to run 5 km every 2 times!” as the advice message AD to the advice request message AR. In this case, upon receiving the advice message AD, the advice processing unit 16 transfers the advice message AD to the user terminal possessed by “Ichiro Tanaka”.

The advice message is not necessarily a natural sentence, and may be, for example, a response message generated by operating a social button (for example, a “like button”).

As described above, it is possible to give advice from other users in the same group to a user who needs advice in the group, and thus, it is possible to promote a behavior change of the entire group.

### Operations and Effects

As described above, according to one embodiment, the following effects can be achieved.

(1) For the group Xi with no dialogue, first, the user xik of which psychological state evaluation value Pij of each user belonging to the group Xi is smaller than the threshold value δpi is extracted as a user who is a behavior change promotion target. Next, a user having a strong relationship with the extracted user having a bad psychological state and having a good psychological state in the group is selected as a user suitable for talking to the user who is a behavior change promotion target. The request message MS for making a request for talking is generated, and the generated request message MS is transmitted to the user terminal UT of the user suitable for talking.

Therefore, the support of the behavior change is provided from the user having a strong relationship with the user in the group to the user having a bad psychological state, and thus the support of the behavior change can be effectively provided to the user who is not in a good condition.

(2) Among the users xij belonging to the group Xi for which it is determined that there is no group dialogue, a user of which goal concreteness Cij is less than the threshold value δci is extracted as a user who is a behavior change promotion target. Another user of which goal concreteness Cik is equal to or more than the threshold value δci is selected in the same group, the behavior change promotion message MS including the text data OD related to the goal and the goal concreteness Cik of the selected user is generated, and the generated behavior change promotion message MS is transmitted to the user terminal UT of the user xij who is a behavior change promotion target.

Therefore, the text data related to the goal and the goal concreteness of the user having the high goal concreteness in the same group are presented to the user having the low goal concreteness in the group, that is, the user of which goal setting is ambiguous, and thus, it is possible to promote the user having the low goal concreteness to review the goal setting.

(3) For the group Xi for which it is determined that there is a dialogue, first, a group in which the dialogue state evaluation value Di is smaller than the dialogue state evaluation threshold value δD, that is, the group Xk in which the dialogue activity (degree of excitement) is low is extracted as a group that is a behavior change promotion target. Next, a group in which the goal group characteristic of each group Xi is similar to that of the group Xk that is a behavior change promotion target and the dialogue state evaluation value Di is equal to or greater than the dialogue state evaluation threshold value δD is selected. That is, a group having similar characteristics of the goal as a group and having a high dialogue activity is selected as a reference group that promotes a behavior change of the group Xk. The behavior change promotion message MS including the text data OD related to the goal and the goal concreteness of the selected similar group is generated, and the generated behavior change promotion message MS is transmitted to the user terminal UT of each user xkj belonging to the group Xk that is a behavior change promotion target.

Therefore, text data related to a goal and goal concreteness of a group having similar goal group characteristics among other groups in which the dialogue activity is equal to or more than the average are presented to each user of the group having a low dialogue activity (degree of excitement). As a result, in a group having a low dialogue activity, it is possible to promote a behavior change as a group with reference to information of another group having a high dialogue activity.

(4) For each group, a user estimated to need advice from other users is selected among users on the basis of the psychological state evaluation value PD and the goal concreteness CD of each user belonging to the group. The advice request message AR for asking another user for advice to the selected user is generated, and the generated advice request message AR is transmitted to the user terminal UT of another user in the group. In a case where the advice message AD is returned from the user terminal UT of another user to the advice request message AR, the advice message AD is transferred to the user terminal UT of the user who needs the advice.

As a result, it is possible to give advice from other users in the same group to the user who needs advice in the group, and thus to promote a behavior change of the entire group.

### Other Embodiments

Although the embodiments of the present invention have been described in detail above, the above description is merely an example of the present invention in all respects. It goes without saying that various improvements and modifications can be made without departing from the scope of the present invention. That is, in carrying out the present invention, a specific configuration according to the embodiment may be appropriately employed.

In short, the present invention is not limited to the above-described embodiments without any change, and can be embodied by modifying the constituents without departing from the concept of the invention at the implementation stage. Appropriately combining a plurality of constituents disclosed in the above embodiment makes it possible to form various inventions. For example, some constituents may be omitted from all the constituents described in the embodiments. The constituents in different embodiments may be appropriately combined.

### REFERENCE SIGNS LIST

SV Server device
G1 to Gm Group
UT User terminal
NW Network
1 Control unit
2 Program storage unit
3 Data storage unit
4 Input interface unit (input I/F unit)
5 Output interface unit (output I/F unit)
6 Bus
11 Relationship data acquisition processing unit
12 Psychological state evaluation processing unit
13 Dialogue state evaluation processing unit
14 Goal concreteness calculation processing unit
15 Behavior change processing unit
16 Advice processing unit
31 Relationship data storage unit
32 Behavior change data storage unit

1. A behavior change support device that supports a behavior change for at least one of a plurality of groups to which a plurality of users belongs, the behavior change support device comprising:
first acquisition processing circuitry that acquires and stores, for each of the groups, relationship information indicating a strength of a relationship between the plurality of users belonging to the group;
second acquisition processing circuitry that acquires, for each of the groups, psychological state evaluation information in which psychological states of the plurality of users belonging to the group are reflected;
first user selection processing circuitry that selects, for each of the groups, a first user of which the acquired psychological state evaluation information does not satisfy a preset first condition as a user who is a support target of the behavior change, and selects, on the basis of the relationship information, a second user of which a strength of a relationship with the first user satisfies a preset second condition as a user who is a request destination of support of the behavior change for the first user; and
first message transmission processing circuitry that generates first message information including information for requesting support of the behavior change, and transmits the generated first message information to a user terminal used by the second user.

2. The behavior change support device according to claim 1, further comprising:
third acquisition processing circuitry that acquires, for each of the groups, personal goal information for the behavior change set by each of the plurality of users belonging to the group, and obtains, for each piece of the acquired personal goal information, a goal concreteness indicating a concreteness of a personal goal;
second user selection processing circuitry that selects, for each of the groups, a third user of which the goal concreteness does not satisfy a preset third condition as a user who is a support target of the behavior change, and selects a fourth user of which the goal concreteness satisfies the third condition as a reference user for supporting the behavior change; and
second message transmission processing circuitry that generates second message information including the personal goal information set by the fourth user as information for supporting the behavior change of the third user, and transmits the generated second message information to a user terminal used by the third user.

3. The behavior change support device according to claim 1, further comprising:
fourth acquisition processing circuitry that acquires, for each of the groups, dialogue state evaluation information in which the degree of activity of dialogue between the plurality of users belonging to the group is reflected;
fifth acquisition processing circuitry that acquires, for each of the groups, personal goal information for the behavior change set by each of the plurality of users belonging to the group, and generates goal group characteristics information indicating features of a goal of the group on the basis of the acquired personal goal information;
third selection processing circuitry that selects a first group in which the dialogue state evaluation information does not satisfy a preset fourth condition as the group that is a support target of the behavior change, and selects a second group in which the goal group characteristics information is similar to that of the first group within a range of a preset fifth condition and the dialogue state evaluation information satisfies the fourth condition as a reference group for supporting the behavior change; and
third message transmission processing circuitry that generates third message information including goal information as the selected second group as information for supporting the behavior change of the first group, and transmits the generated third message information to the user terminals used by the plurality of users belonging to the first group.

4. The behavior change support device according to claim 1, further comprising:
fourth selection processing circuitry that selects a fifth user who is an advice target of the support of the behavior change on the basis of at least the psychological state evaluation information;
fourth message transmission processing circuitry that generates advice request message information for requesting advice related to the fifth user and personal goal information set by the fifth user, and transmits the generated advice request message information to the user terminal used by a sixth user other than the fifth user; and
fifth message transmission processing circuitry that, in a case where response message information for the advice request message information is returned from the user terminal used by the sixth user, receives the returned response message information, generates advice information including the received response message information, and transmits the advice information to the user terminal used by the fifth user.

5. A behavior change support method executed by an information processing device supports a behavior change for at least one of a plurality of groups to which a plurality of users belongs, the behavior change support method comprising:
acquiring and storing, for each of the groups, relationship information indicating a strength of a relationship between the plurality of users belonging to the group;
acquiring, for each of the groups, psychological state evaluation information in which psychological states of the plurality of users belonging to the group are reflected;
selecting, for each of the groups, a first user of which the acquired psychological state evaluation information does not satisfy a preset first condition as a user who is a support target of the behavior change, and selecting, on the basis of the relationship information, a second user of which a strength of a relationship with the first user satisfies a preset second condition as a user who is a request destination of support of the behavior change for the first user; and
generating first message information including information for requesting support of the behavior change, and transmitting the generated first message information to a user terminal used by the second user.

6. A non-transitory computer readable medium storing a program for causing a processor included in the behavior change support device to execute processing of each of the processing circuitries included in the behavior change support device according to claim 1.

7. A non-transitory computer readable medium storing a program for causing a processor to perform the method of claim 5.
